(19) United States
(12) Patent Application Publication
(10) Pub. No.: US 2016/0220358 A1
(43) Pub. Date: Aug. 4, 2016

(54) Delivery Tool For Percutaneous Delivery Of A Prosthesis

(71) Applicant: HLT, Inc., Maple Grove, MN (US)

(72) Inventors: Robert Foster Wilson, Roseville, MN (US); John Gainor, Mendota Heights, MN (US)

(73) Assignee: HLT, Inc., Maple Grove, MN (US)

(21) Appl. No.: 15/096,110

(22) Filed: Apr. 11, 2016

Related U.S. Application Data
(62) Division of application No. 11/864,557, filed on Sep. 28, 2007.
(60) Provisional application No. 60/827,373, filed on Sep. 28, 2006.

(51) Int. Cl. A61F 2/24

(52) U.S. Cl. CPC A61F 2/243; A61F 2/2436

(57) ABSTRACT

An expandable delivery tool for aiding the deployment of a prosthesis device within a patient. The delivery tool has a generally elongated shape with a selectively expandable distal end region that flares outward in diameter. Once advanced percutaneously within a patient's vessel, the delivery device can help locate a target area, assist in deploying a prosthesis at a desired position and further expand the prosthesis after deployment.

### RELATED APPLICATIONS

This application is a divisional of U.S. patent application Ser. No. 11/864,557 filed Sep. 28, 2007 entitled Delivery Tool For Percutaneous Delivery Of A Prosthesis, which claims priority to U.S. Provisional Application Ser. No. 60/827,373 filed Sep. 28, 2006 entitled Delivery Tool For Percutaneous Delivery Of A Prosthesis, both of which are hereby incorporated by reference in their entireties.

### BACKGROUND OF THE INVENTION

There has been a significant movement toward developing and performing cardiovascular surgeries using a percutaneous approach. Through the use of one or more catheters that are introduced through, for example, the femoral artery, tools and devices can be delivered to a desired area in the cardiovascular system to perform any number of complicated procedures that normally otherwise require an invasive surgical procedure. Such approaches greatly reduce the trauma endured by the patient and can significantly reduce recovery periods. The percutaneous approach is particularly attractive as an alternative to performing open-heart surgery.

Valve replacement surgery provides one example of an area where percutaneous solutions are being developed. A number of diseases result in a thickening, and subsequent immobility or reduced mobility, of heart valve leaflets. Such immobility also may lead to a narrowing, or stenosis, of the passageway through the valve. The increased resistance to blood flow that a stenosed valve presents can eventually lead to heart failure and ultimately death.

Treating valve stenosis or regurgitation has heretofore involved complete removal of the existing native valve through an open-heart procedure followed by the implantation of a prosthetic valve. Naturally, this is a heavily invasive procedure and inflicts great trauma on the body leading usually to great discomfort and considerable recovery time. It is also a sophisticated procedure that requires great expertise and talent to perform.

Historically, such valve replacement surgery has been performed using traditional open-heart surgery where the chest is opened, the heart stopped, the patient placed on cardiopulmonary bypass, the native valve excised and the replacement valve attached. On the other hand, a proposed percutaneous valve replacement alternative method is disclosed in U.S. Pat. No. 6,168,614, which is herein incorporated by reference in its entirety. In this patent, the prosthetic valve is mounted within a stent that is collapsed to a size that fits within a catheter. The catheter is then inserted into the patient's vasculature and moved so as to position the collapsed stent at the location of the native valve. A deployment mechanism is activated that expands the stent containing the replacement valve against the valve cusps. The expanded structure includes a stent configured to have a valve shape with valve leaflet supports that together take on the function of the native valve. As a result, a full valve replacement has been achieved but at a significantly reduced physical impact to the patient.

More recent techniques have further improved over the drawbacks inherent in U.S. Pat. No. 6,168,614. For example, one approach employs a stentless support structure as seen in U.S. patent application Ser. No. 11/443,814, entitled Stentless Support Structure, filed May 26, 2006, the contents of which are herein incorporated by reference. The stentless support structure provides a tubular mesh framework that supports a new artificial or biological valve within a patient's vessel. The framework typically exhibits shape memory properties which encourage the length of the framework to fold back on itself at least once and possibly multiple times during delivery. In this respect, the framework can be percutaneously delivered to a target area with a relatively small diameter, yet can expand and fold within a vessel to take on a substantially thicker diameter with increased strength.

Typically, the stentless support structure is delivered at the location of a diseased or poorly functioning valve within a patient. The structure expands against the leaflets of the native valve, pushing them against the side of the vessel. With the native valve permanently opened, the new valve begins functioning in place of the native valve. Optimally placing the stentless support structure involves percutaneously passing the structure through the diseased valve, deploying a distal end of the structure until the distal end flares outwardly, then pulling the structure back through the diseased valve until the user can feel the flared distal end of the structure contact a distal side of the diseased valve. Once confident that the flared distal end of the structure is abutting a distal side of the diseased valve, the remaining portion of the structure is deployed within the diseased valve.

In any of the above mentioned percutaneous valve device implant procedures, a significant challenge to device function is accurate placement of the implant. If the structure is deployed below or above the optimal device position, the native valve leaflets may not be captured by the prosthetic support structure and can further interfere with the operation of the implant. Further, misplacement of the support structure may result in interference between the prosthetic device and nearby structures of the heart, or can result in leakage of blood around the structure, circumventing the replacement valve.

Accurate placement of these devices within the native valve requires significant technical skill and training, and successful outcomes can be technique-dependent. What is needed is a delivery tool for more reliably locating a target deployment area, for positioning a percutaneous aortic valve replacement device or other prosthetic device in which the device location during implantation is critical (e.g., an occluder for vascular atrial septal defects, ventricular septal defects, patent foramen ovale or perforations of the heart or vasculature), and for the subsequent deployment of such a device to provide more reliable implant outcomes.

### SUMMARY OF THE INVENTION

In one embodiment, the present invention provides an expandable delivery tool for deploying a prosthesis device within a patient. The delivery tool has a generally elongated shape with an expandable distal end region that flares outward in diameter.

In one aspect, the delivery tool provides a tactile indication of a desired target area, such as a valve. For example, once expanded within a patient's vessel, the delivery device can be pulled proximally towards the user until it contacts a desired target valve. This contact is transmitted and thereby felt by the user on a proximal end of the device outside the patient, providing an indication that a desired target location has been located.

In another aspect, the delivery tool provides a stationary backstop against which a prosthesis can be deployed, further ensuring the prosthesis is delivered at a desired target location within the patient. For example, the expanded backstop of the delivery tool is positioned at a location just distal to a native valve within a patient. The prosthesis is deployed within the native valve and against the expanded backstop, ensuring the prosthesis maintains its intended target position within the native valve.

In yet another aspect, the delivery tool is used to further expand the prosthesis after deployment. For example, the expandable backstop is reduced in size to a desired expansion diameter (i.e., the diameter the user wishes to expand the prosthesis to), then pulled through the deployed prosthesis, causing the diameter of the prosthesis to expand. This expansion further anchors the prosthesis against the vessel, ensuring its position is maintained and minimal leakage occurs past the periphery of the prosthesis. Alternately, the distal end of the delivery tool can be expanded within the prosthesis to further expand the prosthesis within the patient's vessel.

### BRIEF DESCRIPTION OF THE DRAWINGS

FIG. 1 illustrates a side view of a delivery tool according a preferred embodiment of the present invention;

FIG. 2 illustrates a side view of the delivery tool of FIG. 1;

FIG. 3 illustrates a perspective view of the delivery tool of FIG. 1;

FIG. 4 illustrates a side view of a valve prosthesis according to a preferred embodiment of the present invention;

FIG. 5 illustrates a side view of a locking-pin mechanism connected to a support structure according to a preferred embodiment of the present invention;

FIG. 6 illustrates a magnified side view of the locking-pin mechanism of FIG. 5;

FIG. 7 illustrates a side perspective view of the locking-pin mechanism of FIG. 5;

FIG. 8 illustrates a bottom perspective view of the locking-pin mechanism of FIG. 5;

FIG. 9 illustrates a side view of the delivery tool of FIG. 1;

FIG. 10 illustrates a side view of the delivery tool of FIG. 1;

FIG. 11 illustrates a side view of the delivery tool of FIG. 1, with a valve prosthesis in the initial stage of deployment;

FIG. 12 illustrates a side view of the delivery tool of FIG. 1, with the initial portion of the prosthesis further deployed;

FIG. 13 illustrates a side view of the delivery tool of FIG. 1, with the initial portion of the prosthesis further deployed;

FIG. 14 illustrates a side view of the delivery tool of FIG. 1 and the prosthesis retracted into a simulated valve site;

FIG. 15 illustrates a side view of the delivery tool of FIG. 1 with the prosthesis having been deployed into a simulated valve site;

FIG. 16 illustrates a side view of the delivery tool of FIG. 1 having been relaxed from its expanded configuration;

FIG. 17 illustrates a perspective view of the delivery tool of FIG. 1 with the prosthesis having been fully deployed;

FIG. 18 illustrates a perspective view of the delivery tool of FIG. 1 being drawn within the prosthetic valve;

FIG. 19 illustrates a perspective view of the delivery tool of FIG. 1 drawn into the prosthetic valve and expanded to provide a means for fully seating the device within the native valve;

FIG. 20 illustrates a perspective view of a prosthesis and the delivery tool of FIG. 1;

FIG. 21 illustrates a side view of a prosthesis and the delivery tool of FIG. 1 with the tool having been fully withdrawn from the prosthetic valve;

FIG. 22 illustrates a side view of a preferred embodiment of a delivery tool with mesh formed into an expanded shape constituting an inverted cone;

FIG. 23 illustrates a side view of a preferred embodiment of a delivery tool with mesh formed into a conical cup shape without inversion of the mesh layers;

FIG. 24 illustrates a side view of a preferred embodiment of the delivery tool constructed with a series of superelastic wire loops for location and placement; and

FIG. 25 illustrates a side view of a preferred embodiment of the delivery tool constructed with a series of balloons for location and placement.

### DETAILED DESCRIPTION OF THE INVENTION

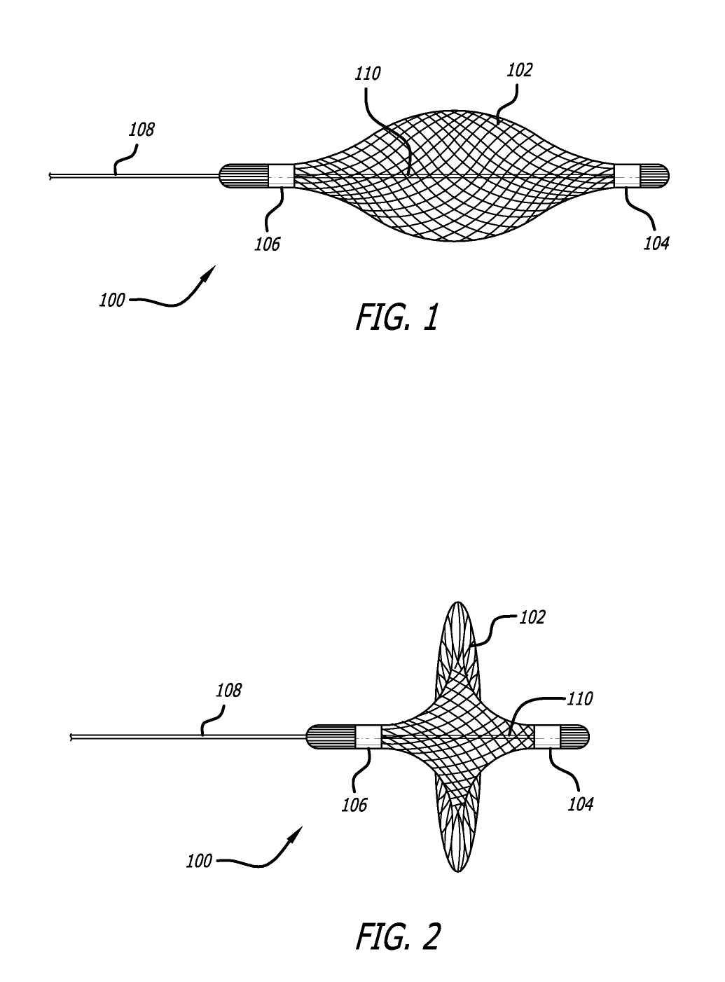

FIG. 1 illustrates an embodiment of an expandable delivery tool 100 according to the present invention. Generally, the expandable delivery tool 100 is removably positioned within the vessel of a patient to assist in the delivery and positioning of a prosthesis at a target area. In this respect, a user can more precisely deploy a prosthesis while minimizing unwanted deployment complications.

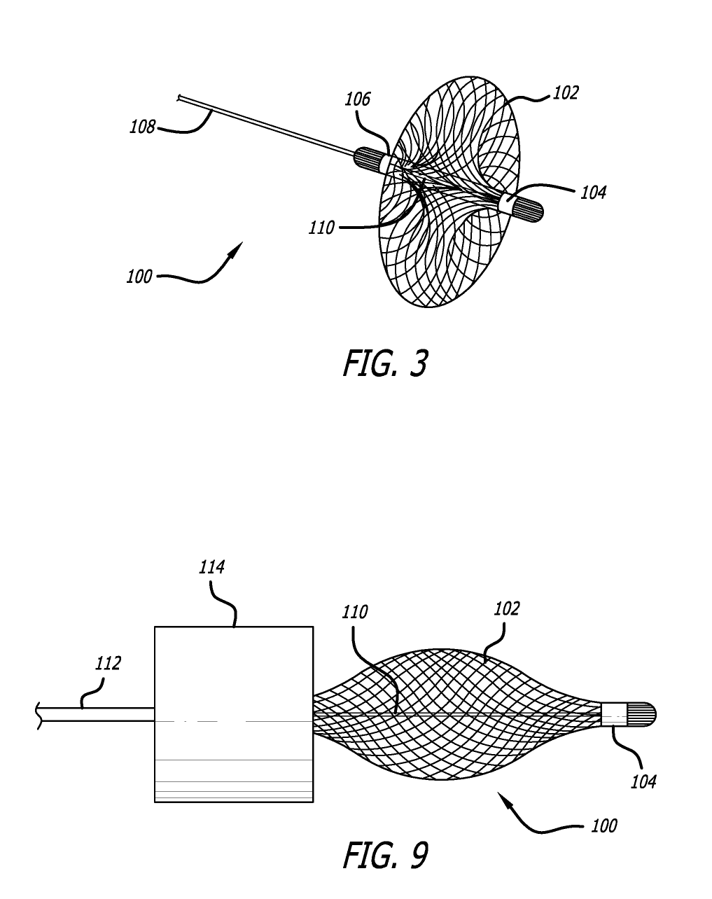

The expandable delivery tool 100 includes a deformable mesh region 102 that expands from a reduced diameter configuration seen in FIG. 1 to a flared expanded diameter configuration seen in FIGS. 2 and 3. The diameter of the mesh region 102 is adjusted by increasing or decreasing the distance between a proximal and distal end of the mesh region 102. More specifically, a distal anchor 104 secures the distal end of the mesh region 102 to a control wire 110 that extends through the mesh region 102 and proximally towards the user. An outer sheath 108 slides over the control wire 110 and is secured to the proximal anchor point 106. Thus, the outer sheath 108 can be moved distally relative to the control wire 110 by the user to increase the diameter of the mesh region 102 and moved proximally relative to the control wire 110 to reduce the diameter of the mesh region 102.

The mesh of the mesh region 102 may be created by braiding together a plurality of elongated filaments to form a generally tubular shape. These elongated filaments may be made from a shape memory material such as Nitinol, however non shape memory materials such as stainless steel or polymeric compounds can also be used. It should be noted that strength and shape of the mesh region 102 can be modified by changing the characteristics of the filaments. For example, the material, thickness, number of filaments used, and braiding pattern can be changed to adjust the flexibility of the mesh region 102.

In a more specific example, the mesh region 102 of each filament has a diameter of 0.008″ and is made from Nitinol wire, braided at 8 to 10 picks per inch. This may result in an included braid angle between crossed wires of approximately 75 degrees.

While mesh is shown for the mesh region 102, other materials or arrangements are possible which allow for selective expansion of this region while allowing profusion of blood past the delivery device 100.

The maximum diameter of the expanded configuration of the mesh region 102 may be increased by increasing the length of the mesh region 102 and therefore allowing the ends of the mesh region 102 to be pulled together from a greater distance apart, or by decreasing the braid angle of the braided Nitinol tube. Similarly, the maximum diameter may be decreased by shortening the length of the mesh region 102 or by increasing the braid angle of the braided Nitinol tube. In other words, the length of the mesh region 102 and the braid angle used will generally determine the maximum expanded diameter that the mesh region 102 may achieve. Thus, the maximum diameter of the mesh region 102 can be selected for a procedure based on the diameter of the target vessel.

In the embodiments shown, the proximal anchor 106 and the distal anchor 104 are metal bands that clamp the mesh region 102 to the outer sheath 108 and control wire 110, respectively. However, other anchoring methods can be used, such as an adhesive, welding, or a locking mechanical arrangement.

The proximal and distal ends of the mesh region 102 may include radiopaque marker bands (not shown) to provide visualization under fluoroscopy during a procedure. For example, these radiopaque bands may be incorporated into the mesh region 102 or may be included with the proximal and distal anchors 106 and 104. In this respect, the user can better observe the position of the mesh region 102 and its state of expansion within the patient.

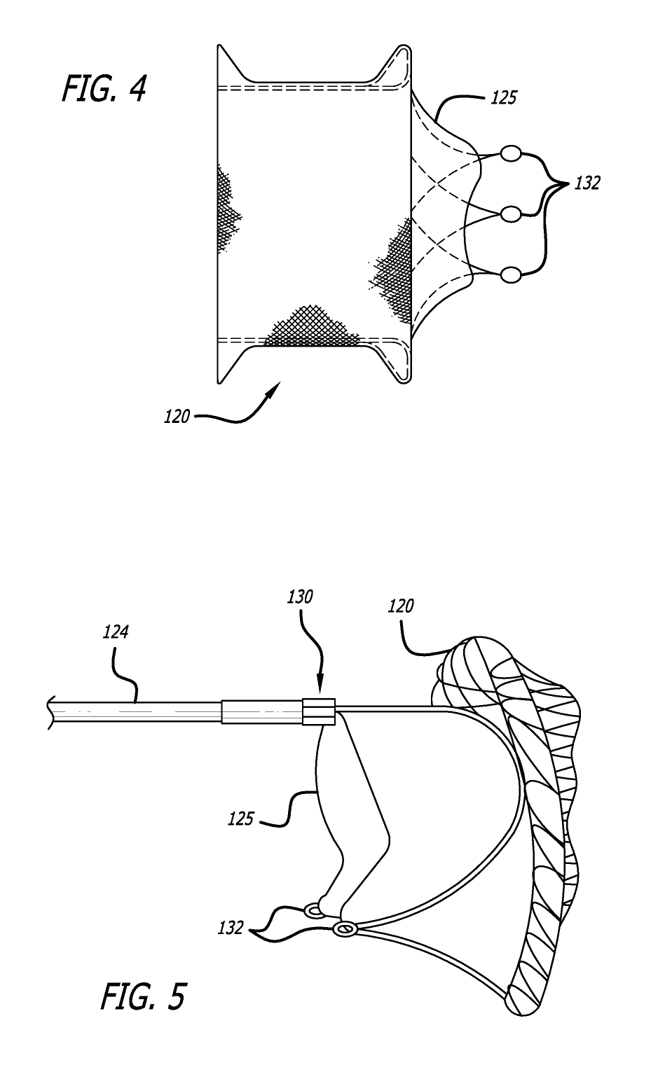

FIG. 4 illustrates an example of a prosthesis that can be delivered and positioned with the delivery device 100. Specifically, the prosthesis is a stentless support structure 120 as seen in U.S. patent application Ser. No. 11/443,814, entitled Stentless Support Structure, filed May 26, 2006, the contents of which are herein incorporated by reference.

As described in the previously incorporated U.S. patent application Ser. No. 11/443,814, the support structure 120 is typically inverted or folded inward to create a multilayer support structure during the delivery. To assist the user in achieving a desired conformation of the support structure 120, the delivery catheter typically includes connection members or arms that removable couple to the eyelets 132 of the support structure 120. In this respect, the user can manipulate the support structure 120, disconnect the connection members and finally, remove the delivery catheter from the patient.

FIGS. 5-8 illustrate a preferred embodiment of a removable coupling mechanism between a connection member 124 of a delivery catheter and the support structure 120. Specifically, a locking-pin mechanism 130, best seen in FIGS. 7 and 8, includes a first jaw member 136 having a locking pin 134 and a second jaw member 138 having an aperture 140 to capture the locking pin 134 when the locking pin mechanism 130 is closed. The jaw members 136 and 138 can be moved between open and closed positions (i.e., unlocked and locked positions) by adjusting control wires (or alternately rods) slideably contained within the connection member 124. The distal ends of the control wires are connected to the jaw members 136 and 138, causing the jaw members 136 and 138 to move near or away from each other.

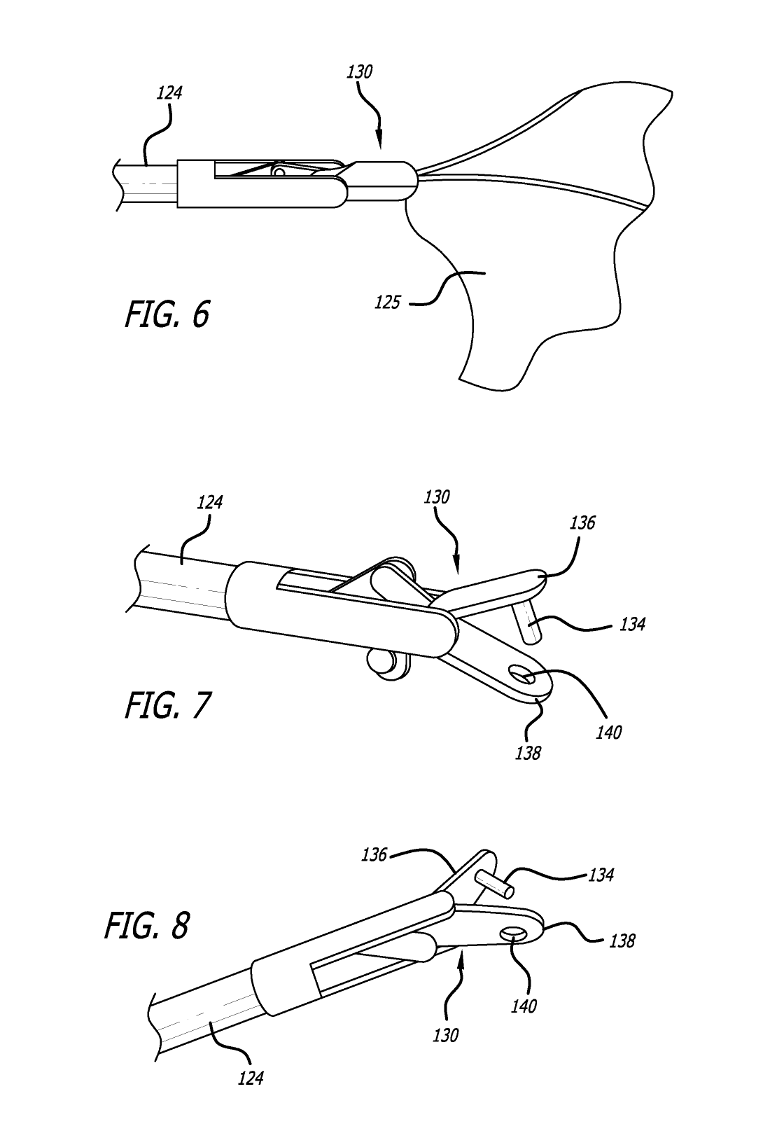

As best seen in FIGS. 5 and 6, the locking-pin mechanism 130 passes through the eyelet 132 of the support structure 120. When the locking-pin mechanism 130 is in the closed position, the eyelet 132 is locked around the connection member 124. When the user wishes to release the support structure 120, the jaw members 136 and 138 are opened allowing the eyelet 132 to slide off of the locking pin 134. In this respect, the user can selectively release the support structure 120 by moving the control wires from a proximal location outside the body.

Preferably, the locking pin 134 has a longitudinal axis that is perpendicular to the longitudinal axis of the connection member 124. Because the locking pin 134 is supported by both jaws 136 and 138 when the mechanism 130 is in the closed position, and because the resulting force placed on the locking pin 134 is normal to the longitudinal axis of the locking pin 134, the locking-pin mechanism 130 is not urged toward the open position when under load. Accordingly, the locking-pin mechanism 130 provides a strong and unbreakable connection with the eyelet 132 until the user disengages the locking-pin mechanism 130 from the eyelet 132 by opening the jaws 136, 138.

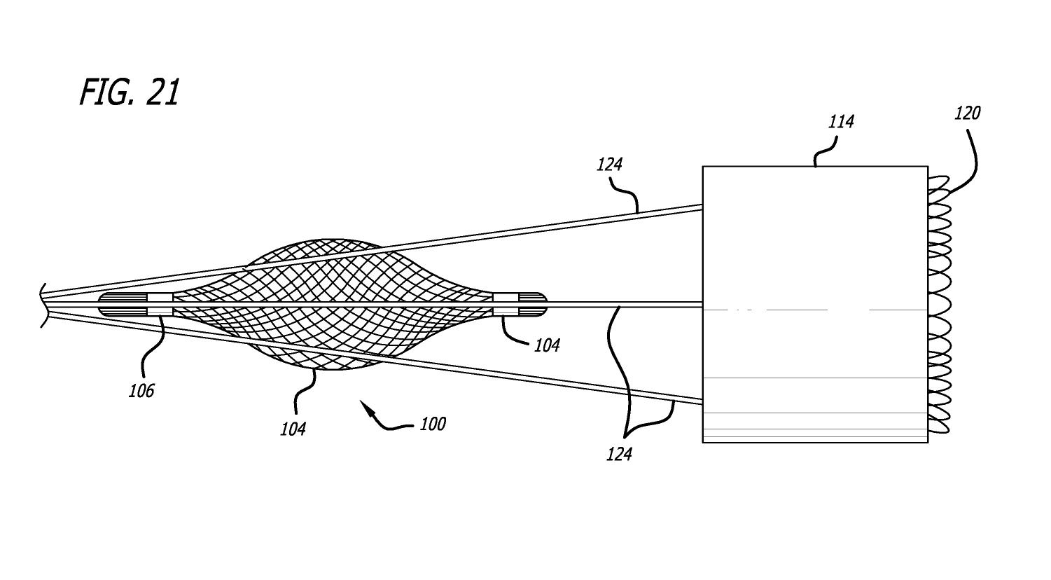

One advantage of the configuration of the connection member 130 and the location of the eyelets 132 is that even when all three connection members 130 are attached to the eyelets 132 (see, e.g., FIG. 21), there is no interference between the connection members 130 and the operation of the valve leaflets 125. Additionally, blood may flow around the delivery mechanism and through the prosthesis. Hence, the operation and location of the prosthesis may be verified prior to release. If the position of the prosthesis is undesirable, or if the valve leaflets 125 are not operating, the prosthesis may be retracted into the delivery mechanism.

Alternately, other coupling mechanisms can be used to retain and release the support structure 120. For example, the connection member 124 may have hooks or breakable filaments at their distal end which allow the user to selectively release the support structure 120.

Operation of the device is now described in detail. Referring to FIGS. 9-21, the delivery tool 100 is illustrated delivering a prosthesis to a piece of clear tubing that represents a native valve 114 (e.g., aortic valve) within a patient. In this example, the prosthesis is the previously described stentless support structure 120. However, it should be understood that the present invention can be used for the delivery of a variety of prosthesis devices including stent devices as seen in the previously discussed Andersen '614 patent, as well as other devices used for occlusion of apertures or perforations of the heart or vasculature.

A distal end of a guidewire and introducer (not shown in the Figures) are typically advanced to the desired target area in the patient's vessel. In this case the target area is a native valve 114. Next, a delivery sheath 112 is slid over the guide catheter until its distal end is at the approximate location of the delivery sheath 112, and the guidewire and introducer are removed.

Referring now to FIG. 9, the delivery tool 100 is advanced through the delivery sheath 112 until the mesh region 102 exits from the distal end of the delivery sheath 112 and passes to a location distal to the target area (i.e., past the target location which in this example is the native valve 114).

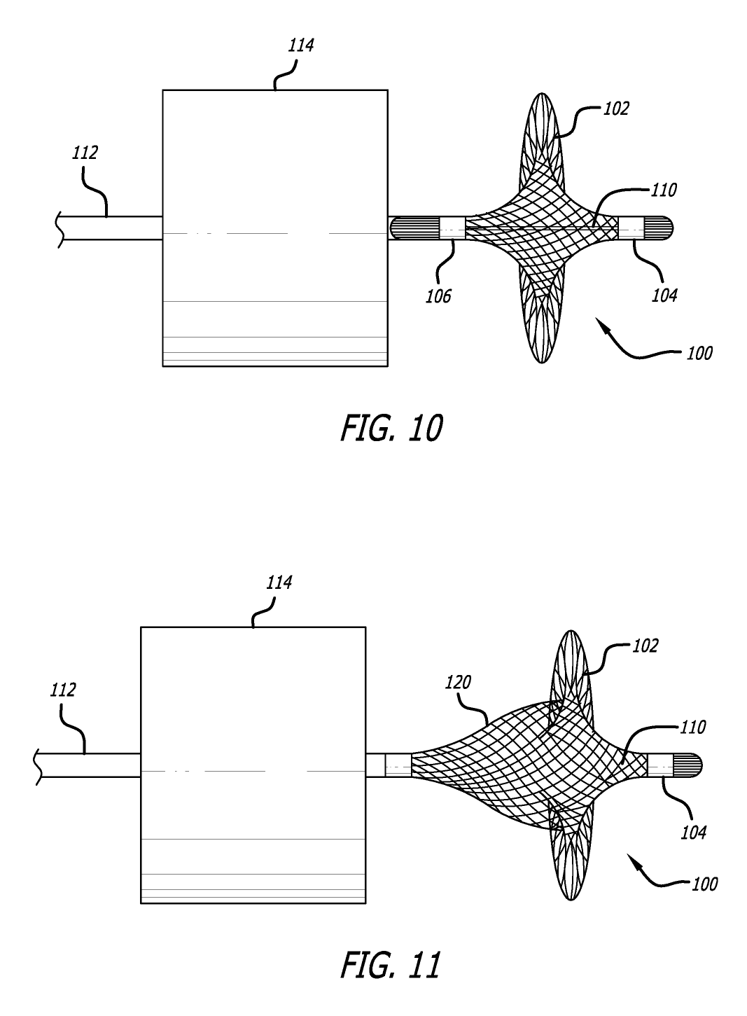

Turning now to FIG. 10, the user moves the delivery tool 100 into its expanded configuration by pulling on the proximal end of the control wire 110 relative to the outer sheath 108. This moves the distal end of the control wire 108 towards the end of the outer sheath 108, compressing the length of the mesh region 102 while increasing or flaring its diameter.

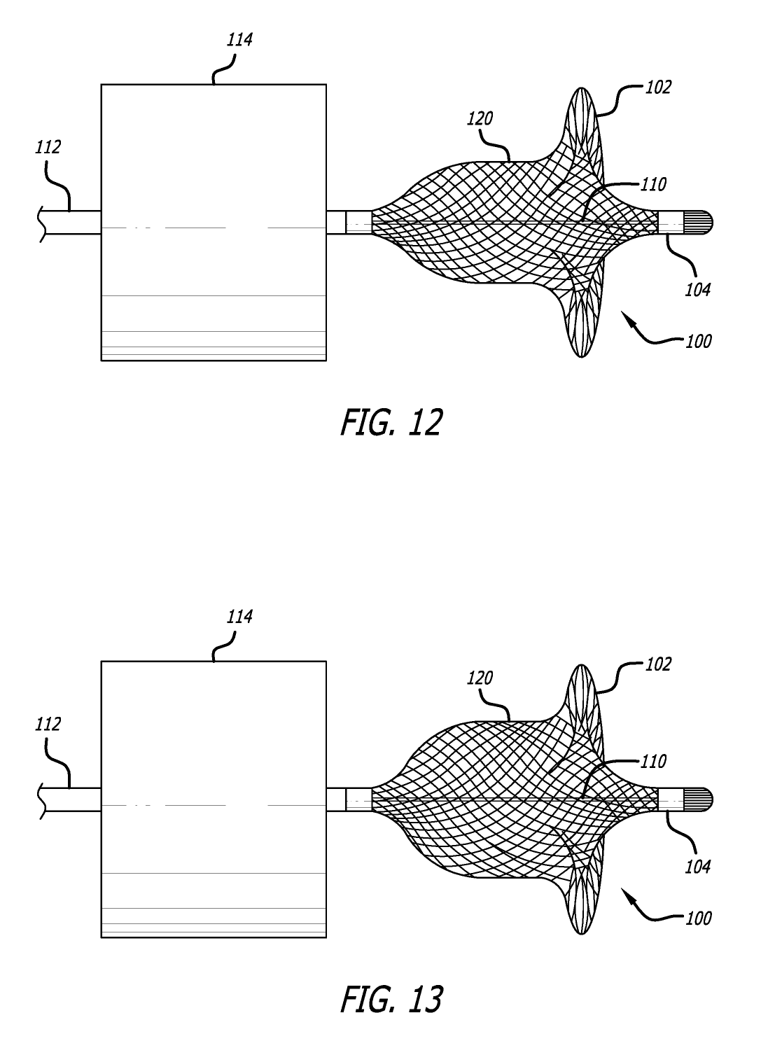

As seen in FIG. 11, a stentless support structure 120 (for anchoring a replacement valve) is advanced out of the distal end of the delivery sheath 112 until it contacts the mesh region 102 of the delivery tool 100. As it continues to advance from the delivery sheath 112, the support structure 120 expands in diameter as seen in FIGS. 12 and 13. In this respect, the support structure 120 becomes at least partially or even fully deployed distally to the native valve 114.

Next, the stentless support structure 120 is advanced from the delivery sheath 112 by multiple connection members 124, seen best in FIGS. 18, 20 and 21. Each of the connection members 124 are removably connected to the stentless support structure 120 at their distal ends and are longitudinally slidable within the delivery sheath 112. In this respect, the user can manipulate a proximal exposed end of the connection members 124 to advance and further position the stentless support structure 120, even after the structure 120 has been partially deployed. Once the stentless support structure 120 has achieved a desired position, and the operation of the prosthesis has been verified, the connection members 124 can be uncoupled from the structure 120 and removed from the patient.

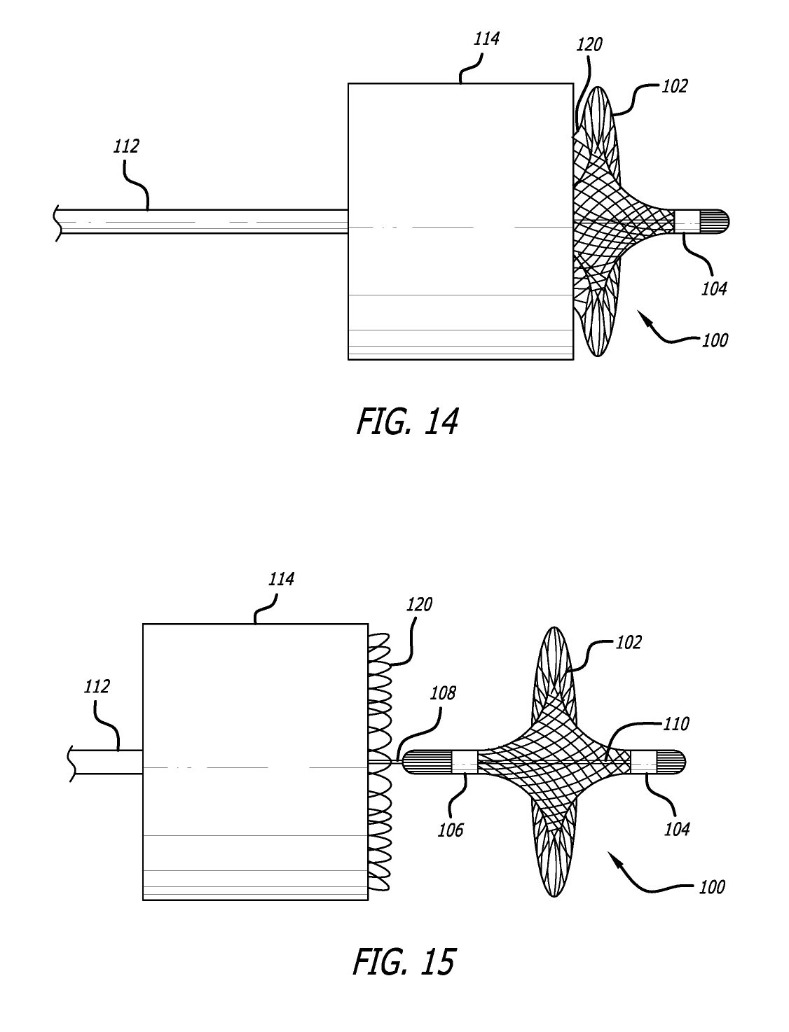

Turning to FIG. 14, both the delivery tool 100 and the stentless support structure 120 are retracted in a proximal direction towards the native valve 114. As the delivery tool 100 retracts, the expanded diameter of the mesh region 102 contacts the native valve 114 to provide the user with a tactile indication. Thus, the user is alerted when the support structure 120 achieves the desired target location within the native valve 114.

As previously described in this application, the stentless support structure 120 is folded inwards on itself to create a dual layer (or even a multiple layer) support structure. This folding configuration allows the stentless support structure 120 to achieve a relatively small delivery profile within the delivery sheath 112 while deploying to have increased wall thickness. While this folding may generally occur by itself due to the preconfigured characteristics of the shape memory material of the support structure 120, additional force in a distal direction may be required to assist the support structure 120 in achieving its final configuration. Typically, this extra force may be generated by advancing the delivery sheath 112 relative to the support structure 120 (i.e., pushing the delivery sheath 112 or by advancing the connection members 124). However, this extra movement by the delivery sheath can dislodge the support structure 120 from the native valve 114, particularly in a distal direction.

To prevent the aforementioned movement of the support structure 120, the expanded mesh region 102 is held in place against the edge of the native valve 114, preventing the support structure 120 from dislodging. In other words, the mesh region 102 of the delivery device 100 acts as a stationary backstop, preventing distal movement of the support structure out of the native valve 114 and therefore allowing the user to more precisely determine the deployed location of the support structure 120 within the patient.

In some circumstances, a user may simply wish to adjust the mesh region 102 to its contracted configuration and remove the delivery device from the patient. In other circumstances, the user may wish to further expand the support structure 120 to provide additional anchoring force against the native valve and to ensure that the leaflets of the native valve remain captured under the support structure 120.

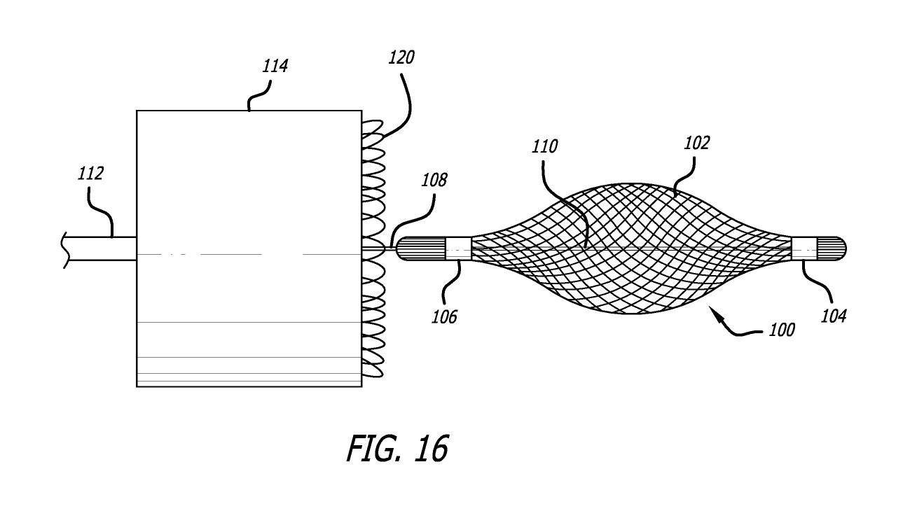

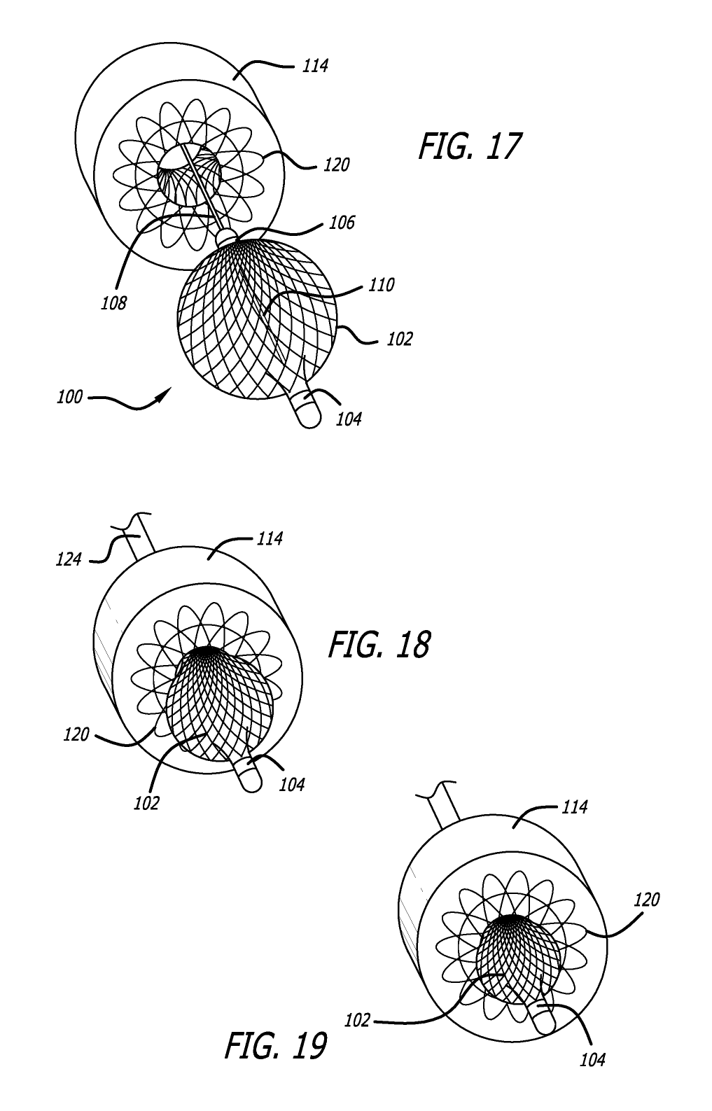

The further expansion of the support structure 120 can be achieved with the mesh region 102 of the delivery tool 100, similar to a balloon catheter. More specifically, the delivery tool 100 is advanced in a distal direction away from the native valve 114, as seen in FIG. 15. As seen in FIGS. 16 and 17, the diameter of the mesh region 102 is reduced to a desired target diameter of the support structure 120 (i.e., the diameter the user wishes to expand the support structure 120 to).

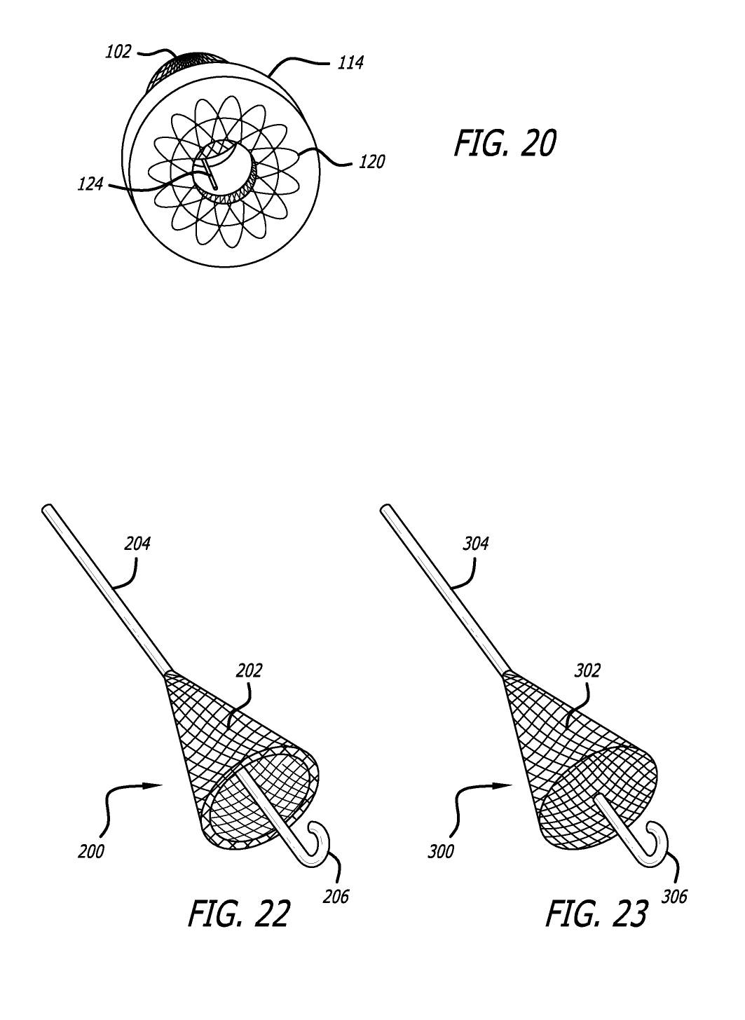

Referring to FIGS. 18 and 19, once the desired diameter of the mesh region 102 has been achieved, the user retracts the delivery device 100 in a proximal direction through the support structure 120 which causes the support structure 120 to further expand against the native valve 114. The resulting expansion of the support structure 120 can be better demonstrated by comparing the perspective view of FIG. 17 to the view shown in FIG. 20.

Once the delivery device has been pulled all the way through the support structure 120 and the native valve 114, as seen in FIG. 21, the mesh region 102 can be further reduced in diameter and removed from the patient. Finally, the connection members 124 can be disconnected from the support structure 120 and removed with the delivery sheath 112.

Alternately, this same expansion of the support structure 120 can be achieved by initially decreasing the diameter of the mesh region 102, positioning the mesh region 102 within the support structure 120, then expanding the mesh region 102 to a desired diameter. Once a desired expansion of the support structure 120 has been achieved, the mesh region 102 can be decreased in diameter and pulled out of the patient.

Other embodiments of the present invention may include a configuration of the mesh region that forms a variety of shapes in the expanded profile and can be used for other applications (e.g., implantable prosthetic devices having similar or different shapes or structures than the support structure 120). For example, FIG. 22 illustrates a delivery device 200 generally similar to the previously described delivery device and further includes an inverted cone shape mesh region 202 connected to an outer sheath 204. In this respect, the mesh region 202 may be selectively expanded to a cone shape for delivery of a support structure.

Additionally, a pig tail 206 can be included on the end of the outer sheath 204 or distal end of the delivery device 200 to act as a bumper, thereby minimizing potential damage that may otherwise be caused by the distal end of the device 200 during delivery. The pigtail may be composed of a short tube composed of a flexible polymer and has a generally curved or circular shape.

In another example, FIG. 23 illustrates a delivery device 300 including a conical cup shaped mesh region 302 which is generally similar to the previously described preferred embodiments 100 and 200. Similarly, the device 300 includes an outer sheath 304 and a pig tail 306 on the distal end of the device 300 to prevent damage to the patient. However unlike the relatively flat distal end of the delivery device 200, the delivery device 300 inverts to form a cup shape having an open, distal end.

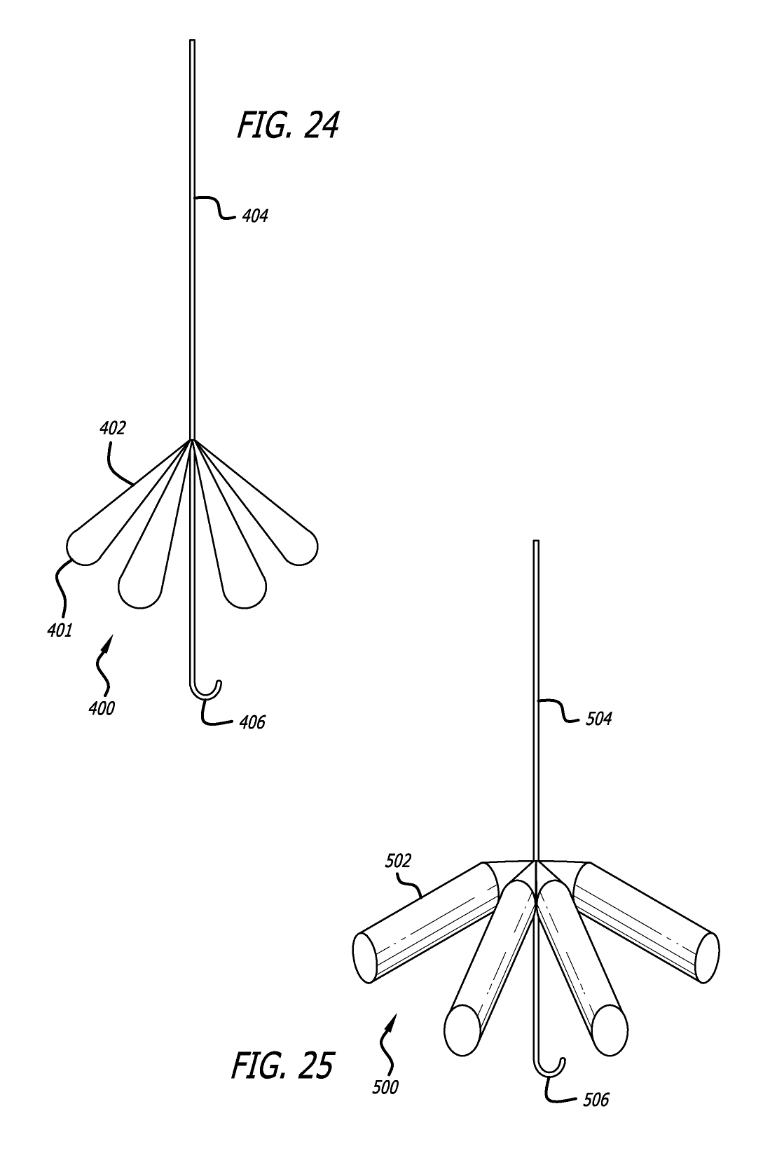

As seen in FIG. 24, a distal end of a delivery device 400 may be constructed with individual arms 401 built from flexible or superelastic wire 402. These arms 401 can be expanded and contracted similar to the previously described embodiments and may also include a pigtail 406 disposed at a distal end of the outer sheath 404 or delivery device 400.

Referring to FIG. 25, a distal end of a delivery device 500 may alternately include a series of expandable balloons 502 linked together to a catheter 504 to provide delivery and positioning functions similar to the previously described embodiment while allowing blood flow through the balloon interstices. The balloons 502 may be inflatable and may be further expandable relative to each other by a mechanism similar to the previously described embodiments. Further, a pigtail may be included on the distal end of the delivery device 500.

While a stentless support structure 120 has been described with regards to the Figures, other prosthesis devices may similarly be delivered with the present invention. For example, the delivery tool 100 may be used to deploy a stent with an attached replacement valve at a poorly functioning target valve. Additionally, this device may be used independently as a tool to perform balloon aortic valvuloplasty or other balloon techniques in which, for example, device porosity and blood flow-through are desired during the procedure.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method of percutaneously delivering a prosthesis comprising:
advancing a distal end of a delivery tool near a target location within a patient;
increasing a diameter of said distal end of said delivery tool;
deploying a prosthesis at said target location, adjacent to said distal end of said delivery tool; and
preventing said prosthesis from advancing past said diameter of said distal end of said delivery tool.

2. The method of claim 1, further comprising:
decreasing said diameter of said distal end of said delivery tool to a desired expanded diameter of said prosthesis; and
moving said distal end of said delivery tool through said prosthesis so as to expand said prosthesis to said desired expanded diameter.

3. The method of claim 1, further comprising:
decreasing said diameter of said distal end of said delivery tool;
moving said distal end of said delivery to within said prosthesis; and
increasing a diameter of said prosthesis by increasing said diameter of said distal end of said delivery tool.

4. The method of claim 1, wherein said increasing a diameter of said distal end of said delivery tool further comprises modifying a configuration of a mesh section of said distal end.

5. The method of claim 1, wherein said advancing a distal end of a delivery tool near a target location within a patient further comprises advancing said distal end of a delivery tool through a valve within a vascular system.

6. A device for delivering a prosthesis within a vascular system, comprising:
an elongated outer sheath having a lumen disposed therethrough;
a control wire disposed within said lumen; and
a mesh member having a first configuration with a first diameter and a second configuration with a second diameter, said second diameter being larger than said first diameter;
wherein relative movement of said control wire relative to said elongated outer sheath deforms said mesh member between said first configuration and said second configuration.
